(19) United States
(12) Patent Application Publication
(10) Pub. No.: US 2006/0116766 A1
(43) Pub. Date: Jun. 1, 2006

(54) Anterior lumbar interbody implant

(76) Inventor: Jean-Philippe Lemaire, La Chapelle (FR)

Correspondence Address: CARL M. NAPOLITANO, PH.D.;ALLEN, DYER, DOPPELT, MILBRATH & GILCHRIST, P.A., 255 SOUTH ORANGE AVE., SUITE 1401, P.O. BOX 3791, ORLANDO, FL, 32802-3791, US

(21) Appl. No.: 11/001,678

(22) Filed: Dec. 1, 2004

(51) Int. Cl. A61F 2/44

(52) U.S. Cl. 623/17.11

(57) ABSTRACT

An intersomatic implant for insertion in the intervertebral space defined between two adjacent lumbar vertebrae, referred to as an overlying vertebra and an underlying vertebra, reestablishes the anatomical shape of the intervertebral space. The implant includes spacer ramps extending between anterior and posterior transverse walls so as to project above a transverse face for determining a relative spacing between the underlying and overlying vertebrae while the implant is being inserted into the intervertebral space. A profile is presented which is circumscribed inside the intervertebral space in which the implant is to be placed.

### CROSS-REFERENCE TO RELATED APPLICATIONS

This application is a continuation of U.S. application Ser. No. 10/275,518, filed Mar. 25, 2003, which itself claims the benefit of International Patent Application No. PCT/FR01/01431, filed Nov. 6, 2002, the disclosures of which are hereby incorporated herein by reference in their entireties, all commonly owned.

### FIELD OF THE INVENTION

The present invention generally relates to intervertebral implants, and in particular to an intersomatic implant for insertion into the intervertebral space after excision of the disk for restoration to an appropriate height between vertebrae and for ensuring bone fusion between said adjacent vertebrae.

More precisely, the invention relates to an intersomatic implant of the lumbar type for placing in an intervertebral space defined between two adjacent lumbar vertebrae after excision of the disk.

### BACKGROUND OF THE INVENTION

In the state of the art, it is known to insert an intersomatic implant in the intervertebral space defined between two adjacent lumbar vertebrae. Numerous embodiments of such implants have been proposed in the prior art. For example, document WO 97/23175 discloses a lumbar intersomatic implant in the form of a cage having two sagittal walls interconnected by an anterior transverse wall and by a posterior transverse wall. Between them, the walls define an open volume suitable for receiving a bone filler substance referred to as a bone graft, that is to come into contact with the spongy bone of each of the vertebral plates, serving to encourage bone fusion between the two vertebrae. That implant also has at least two walls projecting from its top and bottom faces and shaped to become inserted progressively in the spongy bone during insertion of the implant, and then to prevent it from moving.

It should be understood that a bone graft placed inside such an implant generally leaves portions that project relative to the transverse faces of the implant. Unfortunately, while the cage is being implanted in the intervertebral space, rubbing occurs between the implant and the vertebrae, leading to damage to those portions of the bone graft that are situated level with the transverse faces of the implant. As a result such a bone graft is not in contact with the spongy bone of the vertebral plates of the vertebrae after the cage has been implanted.

### SUMMARY OF THE INVENTION

The invention seeks to remedy the above-specified drawbacks by providing an intersomatic implant that is to be inserted in the intervertebral space defined between two adjacent lumbar vertebrae, and designed by means of a bone graft placed inside the implant to enable good bone continuity to be established between the spongy bone of the vertebral plates of the two adjacent lumbar vertebrae.

The intersomatic implant is inserted into the intervertebral space defined between two adjacent lumbar vertebrae, referred to as an overlying vertebra and an underlying vertebra, in order to reestablish the anatomical shape of the intervertebral space. The implant may be in the form of a cage having at least two sagittal walls interconnected at least by an anterior transverse wall and by a posterior transverse wall that are substantially parallel to a front plane. The walls define an open volume between them for bone filler and present rims extending on one side to define a first transverse face and on the other side to define a second transverse face.

An embodiment of the invention may include at least one spacer ramp extending between the anterior and posterior transverse walls so as to project relative to a transverse face in order to determine relative spacing between the underlying and overlying vertebrae while the implant is being inserted into the intervertebral space, and present a profile which is to be found circumscribed inside the intervertebral space in which the implant is to be placed.

Various other characteristics may appear from the following description given with reference to the accompanying drawings that show particular embodiments of the invention as non-limiting examples.

### BRIEF DESCRIPTION OF THE DRAWINGS

For a fuller understanding of the invention, reference is made to the following detailed description, taken in connection with the accompanying drawings illustrating various embodiments of the present invention, in which:

FIG. 1 is a perspective view of an embodiment of an implant in accordance with the present invention;

FIG. 2 is a sagittal view of an implant seen substantially along arrow F.sub.2 of FIG. 1;

FIG. 3 is a transverse view of the implant shown in FIGS. 1 and 2; and

FIGS. 4 and 5 are sagittal views illustrating, respectively, one embodiment of an implant in a position while being implanted, and in a final, implanted position.

### DETAILED DESCRIPTION OF THE PREFERRED EMBODIMENTS

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout, and prime notation is used to indicate similar elements in alternate embodiments.

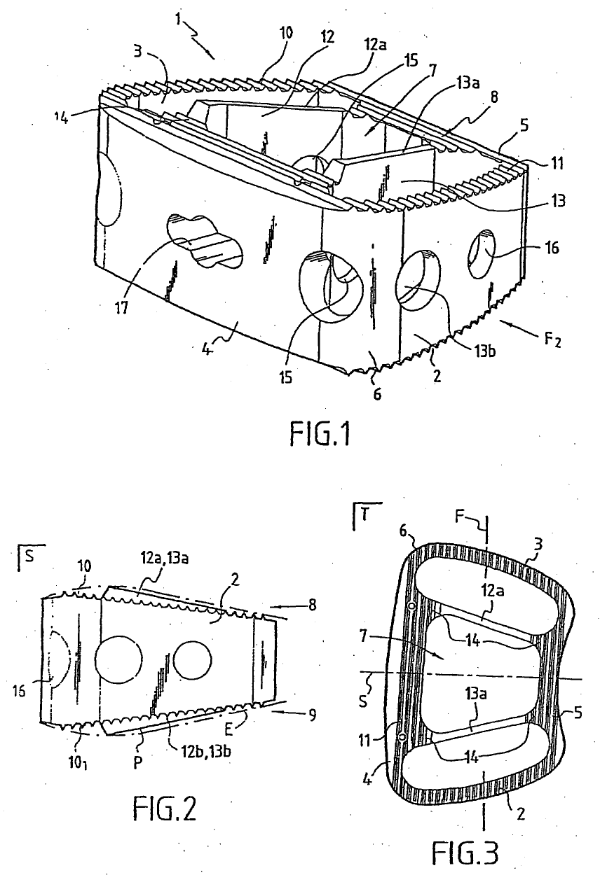

Referring initially to FIGS. 1 to 3, one embodiment of the invention is herein presented as an intersomatic implant and referred to as a cage 1 having a generally rectangular shape for insertion in an intervertebral space after excision of disk material, the intervertebral space being defined as the space between two adjacent lumbar vertebrae and referred to in the description below as the intervertebral space E. With continued reference to FIGS. 1-3, the cage 1 has a first sagittal wall 2 and an opposing second sagittal wall 3 interconnected by an anterior transverse wall 4 and a posterior transverse wall 5. In the example shown, the sagittal walls 2 and 3 extend perpendicularly to a transverse plane T and slope relative to a plane S, referred to as a sagittal or antero-posterior plane that is perpendicular to the plane T. More precisely, the sagittal walls 2 and 3 converge going away from the anterior wall 4. The anterior and posterior transverse walls 4 and 5 extend substantially parallel to each other and to a front plane F perpendicular to the sagittal plane S.

As illustrated herein by way of example, the cage 1 includes connection fillets 6 formed between the sagittal walls 2 and 3 and the transverse walls 4 and 5 at their vertical outside faces so as to provide a cage 1 having corners that are rounded on the outside. As further illustrated with reference to FIG. 3, each sagittal wall 2 and 3, and also the anterior transverse wall 4, possesses convex curvature in the plane T perpendicular to the front plane F, whereas the posterior transverse wall 5 possesses concave curvature so as to leave the vertebral channel free.

On the inside, the cage 1 presents a volume 7 defined by the inside vertical faces of the walls 2 to 5, which volume is to be filled with a bone filler substance referred to as a bone graft and adapted to intersomatic fusion. This volume 7 opens out into a first transverse face 8, the top face in the example shown, and a second transverse face 9, the bottom face in the example shown. The walls 2 to 5 present rims 10 defining the top transverse face 8 and rims 10, defining the bottom transverse face 9.

The cage 1 has serrations 11 formed in the rims 10 and 101 of the walls 2 to 5 so as to enable the cage to engage the overlying and underlying vertebrae. The serrations 11 extend parallel to one another and to the front plane F. In general, it should be understood that the top and bottom transverse faces 8 and 9 corresponding to the envelope containing the tips of the serrations 11.

In accordance with the teaching of the present invention, the cage 1 may further include at least one, and in the example shown four, ramps 12a, 12b, 13a, and 13b each extending between the front and rear transverse walls 4 and 5 and adapted, designed, or shaped so as to serve to hold the vertebrae apart, as can be seen from the description below. Each spacer ramp 12a-13a and 12b-13b projects relative to an adjacent transverse face, respectively the face 8 or the face 9. Relative to the adjacent transverse face 8 or 9, each spacer ramp 12a, 12b, 13a, and 13b is of a profile that increases progressively going towards the anterior transverse wall 4. In the example shown, the spacer ramps 12a, 12b, 13a, and 13b extend perpendicularly to the transverse plane T, sloping relative to the sagittal plane S. More precisely, the spacer ramps 12a and 13a, and the spacer ramps 12b and 13b converge going away from the anterior wall 4, and they are symmetrical about the sagittal plane S.

The spacer ramps 12a, 12b, 13a, and 13b project from the sagittal faces 8 and 9 and converge towards each other towards the posterior transverse wall 5 such that the cage 1 is frustoconical in section in the sagittal plane S, thereby making it possible to define an angle for reestablishing curvature of the spine. Selecting heights for the anterior and posterior transverse walls 4 and 5 makes it possible to provide a range of cages suitable for reestablishing different angles of curvature.

In an advantageous embodiment, each spacer ramp 12a, 12b, 13a, and 13b is connected to each transverse wall 4, 5 via a connection zone 14 extending the transverse wall at the same level as the corresponding portion of the sagittal walls 2, 3. It should be understood that the profiles of these connection zones 14 match the profiles of the correspondingly situated sagittal walls 2, 3. These connection zones 14 present edges that are likewise provided with serrations 11. It should thus be considered that the spacer ramps 12a, 12b, 13a, and 13b are shorter in length than the sagittal walls 2, 3.

In another advantageous embodiment, the ramps 12a, 12b, 13a, and 13b present a profile P which is circumscribed inside the intervertebral space E, as represented by continuous lines in FIG. 2 and in which the implant is to be placed. It should be understood that these spacer ramps which project from the transverse faces 8 and 9 of the implant are dimensioned so as to remain in the intervertebral space E and avoid penetrating into the underlying and overlying vertebrae.

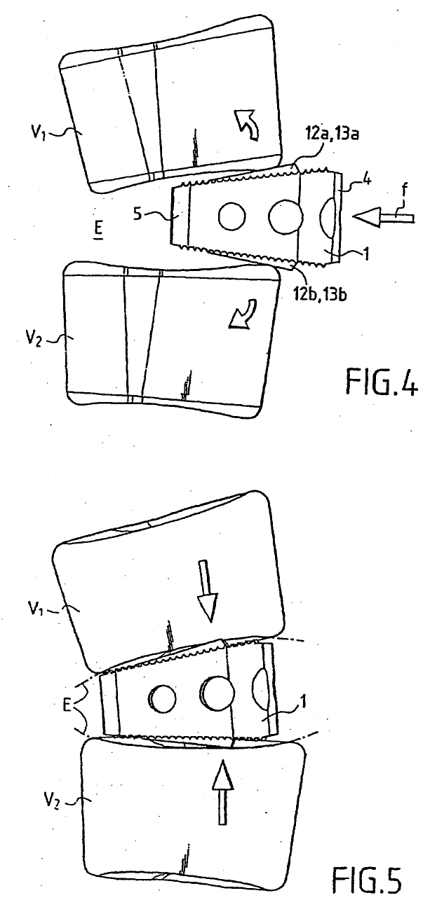

FIGS. 4 and 5 show more explicitly the functions of the spacer ramps of the cage 1 of the invention. The cage 1 is intended to restore the intervertebral space E between two lumbar vertebrae V₁ and V₂ that are shown diagrammatically. As can be seen in FIG. 4, while the implant is being put into place along a path that is initially anterior, the cage 1 is moved in an insertion direction f lying in the sagittal plane S. While the cage 1 is being inserted into the intervertebral space E with the posterior transverse wall 5 situated downstream in the insertion direction, the spacer ramps 12a, 12b, 13a, and 13b serve to move or space apart the overlying and underlying vertebrae V₁ and V₂. The presence of such spacer ramps makes it possible to avoid scraping the overlying and underlying vertebrae V₁ and V₂, thus making it possible to ensure that the overlying and underlying vertebrae V₁ and V₂ do not scrape the bone graft placed in the volume 7 of the cage. Given the profile of the spacer ramps 12a, 12b, 13a, and 13b that increases going away from the posterior transverse wall 5 towards the anterior transverse wall 4, the two vertebrae V₁ and V₂ can be spaced apart progressively. Once the cage 1 is in its final position in the intervertebral space E (FIG. 5), it can be seen that the spacer ramps 12a, 12b, 13a, and 13b remain confined inside the intervertebral space E defined between the overlying and underlying vertebrae. The cage 1 is held between the overlying and underlying vertebrae by the rims of the walls 2 to 5 that bear against the vertebral space. Given that the bone graft placed inside the cage 1 is not expelled in full or in part from the volume 7 while the implant is being put into place, good contact can be obtained between the bone graft and the spongy bone of the vertebral plates of the vertebrae V₁ and V₂.

In the embodiment herein described by way of example, the cage 1 has four spacer ramps 12a, 12b, 13a, and 13b. It should be understood that the spacer ramps 12a and 12b together define a first intermediate wall 12 extending in the same plane that is inclined relative to the sagittal plane S. Likewise, the spacer ramps 13a and 13b together define a second intermediate wall 13 extending in the same plane that is inclined relative to the sagittal plane S. These intermediate walls 12 and 13 preferably extend symmetrically about the sagittal plane S of the implant.

The intermediate walls 12 and 13 thus subdivide the filler volume 7 into three individual volumes. These intermediate walls 12 and 13 preferably have holes 15 allowing communication to be established between the various individual walls defined between them. The sagittal walls 2 and 3 and the anterior transverse wall 4 preferably likewise have through holes 16 encouraging. vascularization of the bone graft. It should be observed that the anterior transverse wall 4 has a slot 17 for receiving a tool that enables the bone graft placed inside the volume 7 to be compacted.

In the example shown, the cage 1 has four spacer ramps 12a, 12b, 13a, and 13b. Naturally, it could be envisaged to make a cage 1 having a single spacer ramp. It could also be envisaged to make a cage having two spacer ramps lying in the same plane. The invention is not limited to the embodiments described and shown, since various modifications can be made thereto without going beyond the ambit of the invention.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

1. An intersomatic implant comprising:
at least two sagittal walls interconnected by an anterior transverse wall and by a posterior transverse wall, wherein the walls in combination define an open volume; and
at least one spacer ramp extending between the anterior and posterior transverse walls so as to project above the first and second opposing transverse faces in order to determine a relative spacing between underlying and overlying vertebrae while the implant is being inserted into an intervertebral space, and wherein the spacer ramp is connected to a transverse wall by a connection zone so that each the at least one spacer ramp is shorter in vertical length than a vertical length of the at least two sagittal walls.

2. An implant according to claim 1, wherein the at least one spacer ramp comprises first and second spacer ramps.

3. An implant according to claim 2, wherein the first and second spacer ramps converge towards each other in a direction towards the posterior transverse wall so as to form a frustoconical section in a sagittal plane in order to define an angle for reestablishing lordosis.

4. An implant according to claim 3, wherein portions of the first and second ramp connecting to interior surfaces of the anterior and posterior walls form rounded corners therewith.

5. An implant according to claim 1, wherein the at least one spacer ramp and the first and second sagittal walls have through holes extending therethrough.

6. An implant according to claim 1, wherein the opposing sagittal walls includes holes extending therethrough, and wherein an opening formed by an anterior most hole is greater the opening in an adjacent posterior hole.

7. An implant according to claims 1, wherein top and bottom surfaces of the sagittal and transverse walls include serrations.

8. An implant according to claim 1, wherein exterior top and bottom edges of the anterior transverse wall are chamfered.

9. An implant according to claim 1, wherein the posterior transverse wall comprises a concave curvature so as to leave a vertebral channel free.

10. An implant according to claim 1, wherein the anterior transverse wall includes a slot sufficient for passing a tool that serves to compact bone filler that is to be placed inside the implant.

11. An implant according to claim 1, wherein a vertical length dimension of the anterior transverse wall is greater than a vertical length dimension of the posterior transverse wall, and wherein the at least two sagittal walls extending therebetween are tapered for a uniform connection and for forming an angle generally having a value of a lordotic angle of adjacent vertebrae for which the implant is contacting.

12. An implant according to claim 12, wherein the opposing sagittal walls converge towards each other in a transverse plane in a direction toward the posterior transverse.

13. An implant comprising:
an anterior transverse wall;
a posterior transverse wall opposing the anterior wall;
a first sagittal wall and an opposing second sagittal wall connected between the anterior and posterior walls for defining an open volume, wherein a vertical length dimension of the anterior transverse wall is greater than a vertical length dimension of the posterior transverse wall, and wherein the first and second sagittal walls extending therebetween are tapered for a uniform connection and for forming a lordotic angle for cooperating with adjacent vertebrae; and
first and second spacer ramps extending between the anterior and posterior transverse walls within the open volume, wherein each of the first and second spacer ramps project outside the open volume proximate the anterior transverse wall ramping toward the posterior transverse wall to within the open volume proximate thereto, and wherein opposing end portions of the first and second spacer ramps include connecting portions for connection to interior surfaces of the anterior and posterior transverse walls that are entirely within the open volume.

14. An implant according to claim 13, wherein the opposing sagittal walls and the two spacer ramps converge towards each other in a transverse plane in a direction toward the posterior transverse.

15. An implant according to claim 13, wherein the first and second spacer ramps converge towards each other in a direction towards the posterior transverse wall so as to form a frustoconical section in a sagittal plane in order to define an angle for reestablishing lordosis.

16. An implant according to claim 13, wherein the connecting portions form rounded corners between surfaces of the spacer ramps and cooperating interior walls of the anterior and posterior transverse walls.

17. An implant according to claim 13, wherein the first and second spacer ramps and the first and second sagittal walls have through holes extending therethrough.

18. An implant according to claim 13, wherein the opposing sagittal walls includes holes extending therethrough, and wherein an opening formed by an anterior most hole is greater the opening in an adjacent posterior hole.

19. An implant according to claims 13, wherein top and bottom surfaces of the sagittal and transverse walls include serrations.

20. An implant according to claim 13, wherein exterior top and bottom edges of the anterior transverse wall are chamfered.

21. An implant according to claim 13, wherein the posterior transverse wall comprises a concave curvature so as to leave a vertebral channel free.

22. An implant according to claim 13, wherein the anterior transverse wall includes a slot sufficient for passing a tool that serves to compact bone filler that is to be placed inside the implant.

23. An implant comprising:
an anterior transverse wall;
a posterior transverse wall opposing the anterior wall;
a first sagittal wall and an opposing second sagittal wall connected between the anterior and posterior walls for defining an open volume, wherein the opposing sagittal walls converge towards each other in a transverse plane in a direction toward the posterior transverse; and
first and second spacer ramps extending between the anterior and posterior transverse walls within the open volume, wherein each of the first and second spacer ramps project outside the open volume proximate the anterior transverse wall ramping toward the posterior transverse wall to within the open volume proximate thereto, and wherein opposing end portions of the first and second spacer ramps include connecting portions for connection to interior surfaces of the anterior and posterior transverse walls that are entirely within the open volume.

24. An implant according to claim 23, wherein a vertical length dimension of the anterior transverse wall is greater than a vertical length dimension of the posterior transverse wall, and wherein the first and second sagittal walls extending therebetween are tapered for forming a lordotic angle for cooperating with adjacent vertebrae.

25. An implant according to claim 23, wherein the first and a second spacer ramps converge towards each other in a transverse plane in a direction toward the posterior transverse.

26. An implant according to claim 23, wherein the first and second spacer ramps converge towards each other in a direction towards the posterior transverse wall so as to form a frustoconical section in a sagittal plane in order to define an angle for reestablishing lordosis.

27. An implant according to claim 23, wherein the connecting portions form rounded corners between surfaces of the spacer ramps and cooperating interior walls of the anterior and posterior transverse walls.

28. An implant according to claim 23, wherein the first and second spacer ramps and the first and second sagittal walls have through holes extending therethrough.

29. An implant according to claim 23, wherein the opposing sagittal walls includes holes extending therethrough, and wherein an opening formed by an anterior most hole is greater the opening in an adjacent posterior hole.

30. An implant according to claims 23, wherein top and bottom surfaces of the sagittal and transverse walls include serrations.

31. An implant according to claim 23, wherein exterior top and bottom edges of the anterior transverse wall are chamfered.

32. An implant according to claim 23, wherein the posterior transverse wall comprises a concave curvature so as to leave a vertebral channel free.

33. An implant according to claim 23, wherein the anterior transverse wall includes a slot sufficient for passing a tool that serves to compact bone filler that is to be placed inside the implant.
